(19) United States
(12) Patent Application Publication
(10) Pub. No.: US 2023/0055777 A1
(43) Pub. Date: Feb. 23, 2023

(54) DEVICE FOR THE PRIMING OF AN EXTRACORPOREAL CIRCUIT

(71) Applicant: EUROSETS S.R.L., Medolla (IT)

(72) Inventors: Antonio PETRALIA, Medolla (IT); Nicola GHELLI, Medolla (IT); Paolo FONTANILI, Medolla (IT)

(73) Assignee: EUROSETS S.R.L., Medolla (IT)

(21) Appl. No.: 17/797,140

(22) PCT Filed: Feb. 15, 2021
(86) PCT No.: PCT/IB2021/051238

§ 371 Date: Aug. 3, 2022

(30) Foreign Application Priority Data
Feb. 18, 2020 (IT) 102020000003308

(51) Int. Cl. A61M 1/36; A61M 39/24

(52) U.S. Cl. CPC A61M 1/3643; A61M 1/3627; A61M 39/24

(57) ABSTRACT

A device comprising a bag made of flexible material and defining a containment volume of a liquid; at least a first duct associated with the bag and provided with a first transit port placed inside the containment volume for the introduction of an operating liquid inside it; at least a second duct associated with the bag and provided with a second transit port placed inside the containment volume for the outflow of the operating liquid to the outside; at least a third duct associated with the bag and provided with a third transit port placed inside the containment volume for the reintroduction of the operating liquid inside it; and at least a fourth duct associated with the bag and provided with a fourth transit port placed inside the containment volume for the outflow to the outside of the air present in the containment volume itself and in the operating liquid.

### TECHNICAL FIELD

The present invention relates to a device for the priming of an extracorporeal circuit.

### BACKGROUND ART

As is well known, in certain surgical operations, during which the functions of the patient's heart are temporarily interrupted, extracorporeal blood circuits are performed using the so-called “heart-lung” machines.

Heart-lung machines comprise a series of devices, including a filtering device (also known as a “venous reservoir”) adapted to filter the incoming blood from the patient, a heat exchanger adapted to regulate the temperature of the blood leaving the filtering device, and an oxygenator adapted to provide the correct supply of oxygen to the blood intended to be pumped back into the patient. In particular, the incoming blood from the patient is sent to the oxygenator by means of a relevant pumping unit.

Before using the extracorporeal circuit it is necessary to carry out what is called, in jargon, the priming of the circuit itself, i.e. its filling by means of a liquid solution in order to remove the air inside.

This operation is generally carried out using a bag that is connected to the various devices of the extracorporeal circuit in order to define a closed circuit, in order to push the air contained inside the circuit itself to the outside. In particular, the bags of this type are provided with a first duct through which the operating liquid is introduced, a second duct adapted to let the liquid flow outwards from the bag in order to flow through the various devices that make up the extracorporeal circuit, a third duct adapted to reintroduce the operating liquid inside the bag, and a fourth duct adapted to allow the air to escape outwards.

In order to prevent the operating liquid from flowing through the circuit also in the opposite direction, a one-way valve is generally positioned outside the bag, along the pipes that carry the operating liquid to the bag itself.

This solution does however have some drawbacks.

In particular, the operating liquid that is introduced into the bag through the first duct can, at the same time, flow out both through the second duct and through the third duct, i.e. in two opposite directions. It follows, therefore, that part of the air contained inside the bag or the ducts connected thereto can be pushed towards the extracorporeal circuit, which must be properly de-bubbled rather than moving away therefrom.

It follows, therefore, that the devices used to date do not ensure effective de-bubbling of the extracorporeal circuit during the priming operations.

### DESCRIPTION OF THE INVENTION

The main aim of the present invention is to devise a device for the priming of an extracorporeal circuit to remove the air contained therein in a convenient and effective manner.

Within this aim, one object of the present invention is to devise a device that is simple to implement and of low cost, as well as safe to operate.

Another object of the present invention is to devise a device for the priming of an extracorporeal circuit that allows overcoming the aforementioned drawbacks of the prior art within a simple, rational, easy, effective to use as well as low cost solution.

The objects mentioned above are achieved by the present device for the priming of an extracorporeal circuit according to claim 1.

### BRIEF DESCRIPTION OF THE DRAWINGS

Other characteristics and advantages of the present invention will become more evident from the description of a preferred, but not exclusive, embodiment of a device for the priming of an extracorporeal circuit, illustrated by way of an indicative, yet non-limiting example, in the accompanying tables of drawings wherein:

FIG. 1 is a front elevation view of a device according to the invention in a first embodiment;

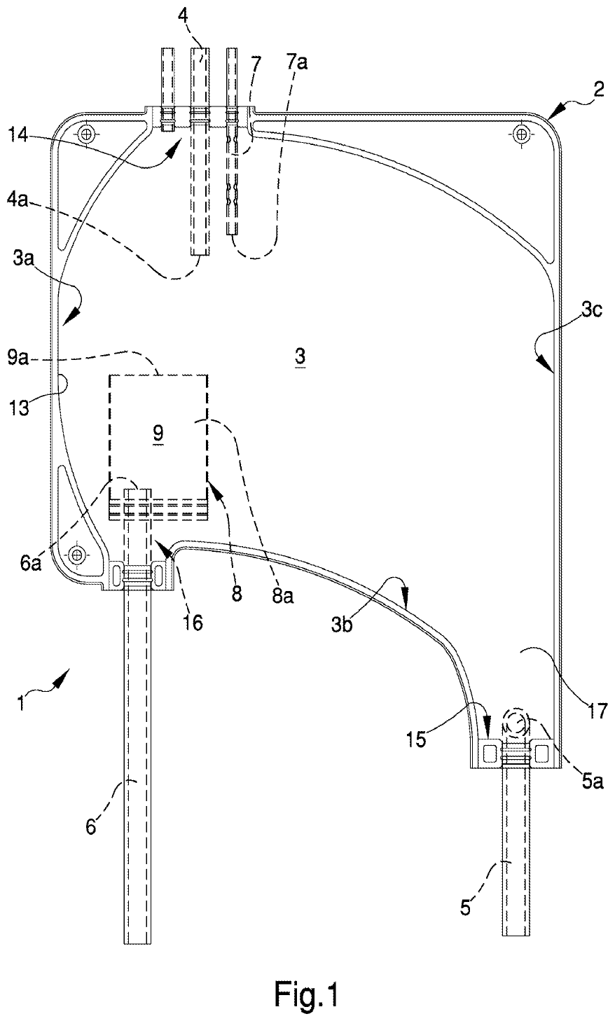

FIG. 2 is a front elevation view of a device according to the invention in a second embodiment.

### EMBODIMENTS OF THE INVENTION

With particular reference to these figures, reference numeral 1 globally indicates a device for the priming of an extracorporeal circuit.

The device 1 comprises a bag 2 made of flexible material and defining a containment volume 3 of a liquid and a plurality of ducts 4, 5, 6, 7 associated therewith, of which at least a first duct 4, at least a second duct 5, at least a third duct 6 and at least a fourth duct 7, provided with a first port 4a, a second port 5a, a third port 6a and a fourth port 7a, respectively, arranged inside the containment volume 3.

In particular, the first port 4a is adapted to allow the introduction of an operating liquid inside the containment volume 3, the second port 5a is adapted to allow the outflow of the operating liquid to the outside, the third port 6a is adapted to reintroduce the operating liquid inside the containment volume 3 and the fourth port 7a allows the outflow of the air present in the containment volume itself and in the operating liquid to the outside.

In the embodiment shown in the figures, the first duct 4 is separate from the second duct 5 and the fourth duct 7.

It cannot however be ruled out that if the first duct 4 is separate from the second duct 5, it may still coincide with the fourth duct 7. In this case, not shown in the figures, the first port 4a through which the operating liquid is introduced inside the containment volume 3 coincides with the fourth port 7a through which the air present in the volume itself is conveyed to the outside. The aforementioned duct is therefore used, alternately, as a duct for the introduction of the operating liquid and as a duct for the outflow of air.

In a further embodiment, not shown in the figures, the first duct 4 coincides with the second duct 5. In this embodiment, the first port 4a coincides, therefore, with the second port 5a. In this case, a fitting, e.g. of the Y type or of the Luer Lock type, is provided along said duct for the introduction of the operating liquid inside the containment volume 3. Also in this case, the aforementioned duct is used, alternately, as a duct for the introduction of the operating liquid inside the containment volume 3 and as a duct for the outflow of the liquid from the containment volume itself.

The second duct 5 and the third duct 6 are connected to each other by interposition of the devices making up the extracorporeal circuit, such as, e.g., a centrifugal pump and an oxygenator. In particular, these devices are connected at inlet to the second duct 5 and at outlet to the third duct 6.

According to the invention, the device 1 comprises one-way valve means 8 associated with the third duct 6, at the point where the third port 6a is located, and adapted to prevent the flow of the operating liquid from the containment volume 3 to the third duct itself. Thus, the valve means 8 are only adapted to allow the operating liquid to flow from the third duct 6 to the containment volume 3 but not vice versa.

Preferably, the valve means 8 are shaped so as to define at least one containment chamber 9 of the third port 6a, wherein such a containment chamber 9 is provided with at least one closable opening 9a which is adapted to allow the transit of the operating liquid flowing out of the third port 6a inside the containment volume 3 but not vice versa.

The third port 6a is then arranged inside the containment chamber 9 defined by the valve means 8.

In other words, the flaps bordering the opening 9a approach each other so as to close the opening itself, thus preventing the flow of the operating liquid, when the latter is outside the containment chamber 9, while they move away from each other, thus defining the opening 9a so as to allow the flow of the operating liquid, when the latter is contained inside the containment chamber 9.

More particularly, the valve means 8 are of the type of a bag made of flexible material, which defines the containment chamber 9 and the opening 9a. The third port 6a is then enveloped by the bag defined by the valve means 8.

In a particular embodiment, the one-way valve means 8 comprise at least two sheets 8a made of a flexible material and sealed together along at least three sides contiguous to each other, through one of which the third duct 6 passes. The non-sealed side of the sheets 8a defines the closable opening 9a.

As can be seen from the figures, the opening 9a is substantially facing the third port 6a.

Advantageously, the device 1 further comprises de-bubbling means 10 of the air contained in the operating liquid placed inside the containment volume 3.

Appropriately, the de-bubbling means 10 are placed around, i.e. surrounding, the one-way valve means 8.

In more detail, the de-bubbling means 10 define a de-bubbling chamber 11, the walls of which are permeable to air and liquids, inside which both the valve means 8 and the fourth port 7a are arranged.

The de-bubbling means 10 are made e.g. of a micro-perforated mesh adapted to facilitate the escape, through the fourth duct 7, of the air still present in the operating liquid that re-enters inside the containment volume 3 through the third duct 6.

For example, the bag 2 is made of two sheets of flexible material overlapping each other and sealed, or otherwise mutually made integral, so as to define the containment volume 3. The containment volume 3 is then bordered by a plurality of perimeter edges defined by the union of the two overlapping sheets. Preferably, the containment volume 3 has a maximum cross-section, detected in a transverse direction to the longitudinal extension of the ducts 4, 5, 6, 7, at the point where the opening 9a is located.

In particular, the containment volume 3 is bordered by at least a first lateral edge 3a that defines a concavity 13 facing inwards the containment volume itself. In other words, the first lateral edge 3a is shaped so as to define an “overhang” facing outwards.

Appropriately, as visible from the figures, the first lateral edge 3a has an at least partly curved extension.

As can be seen from the figures, the first duct 4, the second duct 5 and the third duct 6 are positioned, respectively, at a first zone 14, at a second zone 15 and at a third zone 16 of the bag 2, wherein the first zone 14 is placed in the upper portion of the bag 2 and wherein the second zone 15 and the third zone 16 are placed in the lower portion of the bag itself. In turn, the fourth duct 7 is also placed at the point where the first zone 14 is located. The terms “upper” and “lower” used herein relate to the position in which the bag is commonly placed during its use.

More particularly, the first lateral edge 3a connects the first zone 14 and the third zone 16 to each other.

Advantageously, the second zone 15 is arranged at a lower level than the third zone 16 and so also the second port 5a is arranged at a lower level than the third port 6a.

Preferably, the containment volume 3 is also bordered by at least one substantially curved bottom edge 3b connecting the third zone 16 to the second zone 15. Since, as described above, the second zone 15 is arranged at a lower level than the third zone 16, the bottom edge 3b has a descending pattern as it proceeds close to the second zone 15.

The containment volume 3 is bordered, then, by a second lateral edge 3c, arranged at the point where the second zone 15 is located, on the opposite side of the first lateral edge 3a; the second lateral edge 3c and a section of the bottom edge 3b define a collecting column 17 of the operating liquid arranged on top of the second port 5a. The collecting column 17 has a significantly smaller cross section than the aforementioned maximum cross section.

The operation of the present invention is as follows.

After the extracorporeal circuit, and therefore the relevant devices that make it up, have been connected to the second and third ducts 5 and 6, the operating liquid is introduced inside the bag 2 through the first duct 4.

The operating liquid entering the inside of the containment volume 3 is conveyed as it progresses towards the second port 5a so as to flow through the second duct 5. More particularly, the operating liquid collects in the collecting column 17.

The valve means 8 prevent the operating liquid that is introduced inside the containment volume 3 from entering the inside of the third duct 6 through the third port 6a. More particularly, due to the presence of the operating liquid outside the containment chamber 9 the opening 9a closes, thus preventing the flow of the operating liquid itself.

The operating liquid then flows entirely through the second port 5a, entering the second duct 5, so as to reach the extracorporeal circuit and the relevant devices that make it up.

Once through the extracorporeal circuit, which is then filled with the operating liquid, the latter enters inside the third duct 6 re-entering inside the containment volume 3 through the third port 6a.

In more detail, the operating liquid flowing out of the third port 6a is initially contained inside the containment chamber 9 defined by the valve means 8 and then flows out thereof through the opening 9a, thereby flowing inside the containment volume 3.

The liquid that is returned to the inside of the containment volume 3 through the third duct 6 carries with it the air which was contained inside the ducts themselves and inside the extracorporeal circuit and which is expelled outwards through the fourth duct 7.

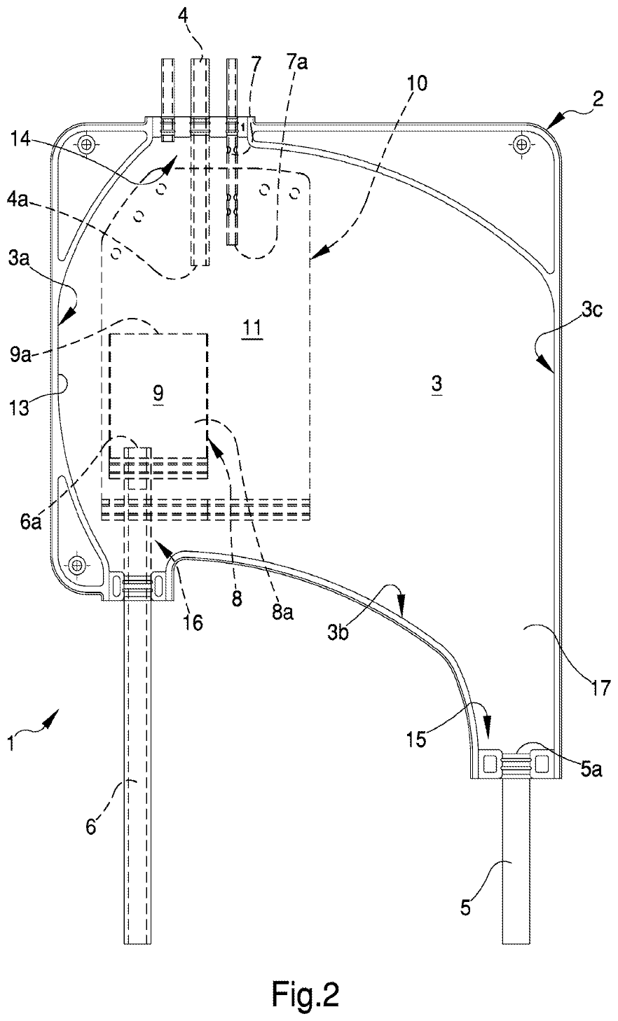

In the second embodiment shown in FIG. 2, the operating liquid flowing out of the valve means 8 is contained inside the de-bubbling chamber 11 in order to facilitate the outflow of air, and in particular of micro-bubbles, through the fourth port 7a.

It has in practice been ascertained that the described invention achieves the intended objects and, in particular, it is emphasized that the device to which the present invention relates allows, thanks to the presence of the one-way valve means arranged to surround the third port, ensuring in an autonomous manner, i.e. without the aid of further devices, that the operating liquid follows the preferential path to remove the air contained in the extracorporeal circuit, thus avoiding its entry inside the third duct.

In addition, the presence of the de-bubbling means arranged to surround the one-way valve means facilitates the outflow of the micro-bubbles present in the operating liquid.

1. A device for the priming of an extracorporeal circuit, the device comprising:
a bag made of flexible material and defining a containment volume of a liquid;
at least a first duct associated with said bag provided with a first transit port placed inside said containment volume for the introduction of an operating liquid inside the containment volume;
at least a second duct associated with said bag provided with a second transit port placed inside said containment volume for the outflow of the operating liquid to the outside;
at least a third duct associated with said bag and provided with a third transit port placed inside said containment volume for the reintroduction of the operating liquid inside the containment volume;
at least a fourth duct associated with said bag and provided with a fourth transit port placed inside said containment volume for the outflow to the outside of the air present in the containment volume itself and in the operating liquid; and
one-way valve means associated with said third duct, at the point where said third port is located, and adapted to prevent the transit of the operating liquid from said containment volume to said third duct.

2. The device according to claim 1, wherein said one-way valve means define at least one containment chamber of said third port, said containment chamber being provided with at least one closable opening adapted to allow the transit of the operating liquid flowing out of said third port inside said containment volume.

3. The device according to claim 2, wherein said one-way valve means are of the type of a bag made of flexible material defining said containment chamber and said closable opening.

4. The device according to claim 2, wherein said one-way valve means comprise at least two sheets made of flexible material and sealed together along at least three sides.

5. The device according to claim 2, wherein said closable opening is substantially facing said third port.

6. The device according to claim 1, further comprising: de-bubbling means of the air contained in the operating liquid placed inside said containment volume.

7. The device according to claim 6, wherein said de-bubbling means are placed around said one-way valve means.

8. The device according to claim 7, wherein said de-bubbling means define a de-bubbling chamber, the walls of which are permeable to air and liquids, said one-way valve means and said fourth port being placed inside said de-bubbling chamber.

9. The device according to claim 1, wherein said first duct coincides with said second duct, said first port coinciding with said second port.

10. The device according to claim 1, wherein said first duct separate from said second duct.

11. The device according to claim 10, wherein said first duct coincides with said fourth duct, said first port coinciding with said fourth port.

12. The device according to claim 1, wherein said containment volume has a maximum cross-section, transversely to the longitudinal extension of said ducts, in the proximity of said opening.

13. The device according to claim 9, wherein said bag comprises at least a first lateral edge, delimiting at least part of said containment volume and defining a concavity facing inwards from the containment volume itself.

14. The device according to claim 10, wherein said first lateral edge has an at least partly curved extension.

15. The device according to claim 1, wherein said first duct, second duct and third duct are placed, respectively, at a first zone, at a second zone and at a third zone of said bag, where said first zone is placed in the upper portion of said bag and where said second zone and third zone are placed in the lower portion of said bag.

16. The device according to claim 10, wherein said first lateral edge connects said first zone to said third zone.

17. A device comprising:
a bag made of flexible material and defining a containment volume of a liquid;
at least a first duct associated with said bag and provided with a first transit port placed inside said containment volume for the introduction of an operating liquid inside the containment volume;
at least a second duct associated with said bag and provided with a second transit port placed inside said containment volume for the outflow of the operating liquid to the outside;
at least a third duct associated with said bag and provided with a third transit port placed inside said containment volume for the reintroduction of the operating liquid inside the containment volume;
at least a fourth duct associated with said bag and provided with a fourth transit port placed inside said containment volume for the outflow to the outside of the air present in the containment volume itself and in the operating liquid; and
a one-way valve associated with said third duct, at the point where said third port is located, and adapted to prevent the transit of the operating liquid from said containment volume to said third duct.
